(19) United States
(12) Patent Application Publication
(10) Pub. No.: US 2022/0354684 A1
(43) Pub. Date: Nov. 10, 2022

(54) APPARATUS FOR CONTINUOUSLY REMOVING GAS THROUGH A NATURAL OR ARTIFICIAL OPENING IN THE BODY OF A PATIENT

(71) Applicant: Advanced Medical Balloons GmbH, Waghäusel (DE)

(72) Inventor: Fred Göbel, Speyer (DE)

(21) Appl. No.: 17/641,685

(22) PCT Filed: Jan. 16, 2020
(86) PCT No.: PCT/IB20/50337

§ 371 Date: Mar. 9, 2022

(30) Foreign Application Priority Data
Sep. 10, 2019 (DE) 10 2019 006 377.5

(51) Int. Cl. A61F 5/441; A61M 1/00

(52) U.S. Cl. CPC A61F 5/441; A61M 1/70; A61M 2205/7536

(57) ABSTRACT

The present invention relates to an apparatus for removing, in a manner that is as odor-free as possible, intestinal gas or other malodorous gases as part of a system for the closed, preferably continuous, removal and/or collection of stools or other strongly odor-forming excretions or secretions from the human or animal body, wherein the stool or the removed excretions or secretions are guided outside the body through a hose-like or tube-like structure which, in addition to an inflow port and an outflow port and a passage region that connects said ports and is intended for the passage of stool or other excretions or secretions, has a branch where intestinal gas or other malodorous gases are separated from the stool or the other excretions or secretions and are conducted through an adsorption and/or filter device to an outlet, wherein at least some regions of the adsorption and/or filter device are spaced apart from the passage region in order to prevent, as far as possible, close contact with the stool or other excretions or secretions flowing through the passage region.

### REFERENCE TO PENDING PRIOR PATENT APPLICATIONS

This patent application claims benefit of International (PCT) Patent Application No. PCT/IB2020/050337, filed 16 Jan. 2020 by Advanced Medical Balloons GmbH for APPARATUS FOR CONTINUOUSLY REMOVING GAS THROUGH A NATURAL OR ARTIFICIAL OPENING IN THE BODY OF A PATIENT, which patent application, in turn, claims benefit of German Patent Application No. DE 10 2019 006 377.5, filed 10 Sep. 2019.

The two (2) above-identified patent applications are hereby incorporated herein by reference.

### FIELD OF THE INVENTION

The present invention describes an apparatus for removing, in a manner that is as odor-free as possible, intestinal gas or other malodorous gases, as part of a system for the closed, preferably continuous, removal and/or collection of stools or other strongly odor-forming excretions or secretions from the human or animal body, wherein the stool or the removed excretions or secretions is or are guided outside the body through a hose-like or tube-like drainage line downstream to a collection container.

### BACKGROUND OF THE INVENTION

Apparatuses for continuously removing stool from the rectum of a patient are an established part of modern medical or nursing care. The stool-removing systems that are know today comprise in a typical construction a head unit designed in a torus-like or balloon-like manner, which anchors (retains) the respective apparatus in the rectum of the patient. The head unit moreover has a hose component that receives the stool in the rectum and channels through the anus or the anal sphincter muscle, on which the torus element or balloon element, which retains the drainage, is distally terminally fixed. The head unit, which is rectally anchored and removes the stool transanally, is attached to another extracorporal hose segment, which transmits the stool from the patient into a generally bag-shaped collection vessel, which is typically placed at a level beneath the patient on the bed frame, whereby the stool drains following a slope. The bag is normally speaking replaceable and has an appropriate manually connecting closure system for attachment to the drainage unit. The connection can be designed for example as a bayonet-like closure. The stool-collecting bag normally comprises a degassing function or component, which discharges intestinal gas that develops in the patient's intestine from the drainage system into the environment or prevents an accumulation of intestinal gas in the abdomen, in that it decompresses the draining system continuously towards the environment. The sufficient decompression of intestinal gas has a high level of clinical relevance in course of the application, because intestinal gases potentially back up as far as the high small intestine and can trigger ileus-like symptoms. An accumulation-induced meteorism can also lead to impairments in diaphragm mobility and thus adversely affect the mechanical ventilation of the patient. The degassing function or component of the bag is frequently equipped with an adsorber or filter, which absorbs the fecal, fetid or other unpleasantly smelling odorous substances.

Stool drainages of a conventional design type are frequently unable to ensure a sufficient decompression or odor adsorption in the case of large quantities of gas to be removed or especially intensive smelling stools. This is caused as a rule by an inadequate quantity of adsorbing or filtering material, but also by the typical positioning of the gas-discharging component within the stool-collecting area of the collection bag. The degassing function of a filter integrated in the bag wall can by be negatively impacted by a transient, flat bearing of the bag, such as frequently occurs for example during transport or positioning of the patient. In a horizontal position of the bag, the contents of the bag reach the gas-outletting component and contaminate or wet the inside thereof, which can restrict the capacity for the decompression of the entire system. The filter or adsorber material that is respectively used can also get engorged with liquid and thus permanently lose its gas-discharging, decompressing function.

### SUMMARY OF THE INVENTION

The described disadvantages of the prior art resulted in the problem that initiated the invention, to ensure, as a part of a generic apparatus for draining and/or collecting stool or other strongly odor-forming excretions or secretions from the human or animal body, that ideally no negative impairment from odors originate from this apparatus.

The solution to this problem is achieved in the case of a generic apparatus by a hose-like or tube-like structure inserted upstream of the collection compartment of the collection container, which structure, in addition to an inflow port and an outflow port and a passage region that connects said ports and is intended for the passage of stool or other excretions or secretions, has a branch where intestinal gas or other malodorous gases are separated from the stool or the other excretions or secretions, and are conducted through an adsorption and/or filter device to an outlet.

Therefore, the invention describes an apparatus for the adsorption and/or filtering of strong-smelling gases formed in the body, which is integrated for example in a system for the continuous closed removal and collection of stool, or even for the removal of other, strongly odor-forming excretions or secretions. The apparatus is designed in particular for a continuously efficient degassing function that is independent of positioning. The removed excretions or secretions are guided in the process within the apparatus in such a way through the tube-like, hose-like or T-piece-like basic element of the apparatus that in the case of a roughly normal vertical position of the apparatus, a contamination or an occlusion of the components discharging gas into the environment can be prevented to a great extent, wherein in particular accumulations of for example intestinal gas within the system can be prevented. Optionally, the apparatus can be permanently integrated into the system, or even be installed in the system by the user as need be as an accessory unit that can be connected in a gas-tight manner. The apparatus preferably has a separating layer from the medium aimed at adsorbing and/or filtering, which repels liquid and/or stool, but is permeable for gas. The adsorbing and/or filtering medium is optionally integrated into the apparatus in a fixed or detachable, replaceable design. The apparatus is positioned between the removing catheter and the collecting bag unit; the contents of the bag are thus not exposed continuously. The collecting bag has a backflow-inhibiting function, which keeps material that has already reached the bag there, even if the bag gets into a flat horizontal position from the normal vertical position. The apparatus moreover ensures that gas that penetrates into the collecting area of the vessel overcomes a backflow-inhibiting function integrated into the vessel and is conveyed to the gas-discharging apparatus that is upstream from collecting vessel.

The apparatus described as a part of the invention for discharging quantities of gas that that formed in the body comprises, in the preferred embodiment, a basic element in the manner of a T-piece, wherein the upper, distal end pointing towards the patient is directly opposite in an axial extension from the lower, proximal end of the T-piece pointing towards the collection bag. In the preferred embodiment, the free, open leg of the T-piece pointing laterally toward the environment receives the odor-adsorbing and/or filtering unit that is filled with a corresponding substance, for example on the basis of activated carbon. In addition to surface-active substances that adsorb the odorous substances accumulating on the surface, absorbing substances that absorb the odor-active substances in the manner of a physical “solution” are likewise possible. The unit can be connected in a fixed design to the lateral leg, or be connected thereto by a corresponding connection, for example mounted.

It is essential for the function of the invention that, in the case of the normal vertical positioning of the degassing component in the stool-removing system, intestinal gas is able to escape from the system unhindered and also efficiently in the case of large quantities of gas. If there is a change in the normal vertical position of the component to a transient horizontal position, a potential occlusion of the components required for the degassing due to stool should be prevented as effectively as possible. As a part of the invention, the adsorbing and/or filtering material is separated by different structural features from the stool removed in the hose line and collected in the bag. The invention describes a basic structural form of the apparatus, in which content that has already been conducted into the collection container or collection bag is impeded from a backflow to the degassing component in the case of a horizontal position, for example due to foil-based, return-inhibiting, lip-like or sail-like components, and the component for degassing is in principle structurally upstream from the collection container.

It is within the scope of the invention that the longitudinal direction of the flow channel between the branch and the adsorption and/or absorption and/or filter device encloses with the longitudinal direction of the passage region between the branch and the outflow port an angle of 90° or less than 90°, for example an angle of 75° or less, preferably an angle of 60° or less, in particular an angle of 45° or less, or even an angle of 30° or less. If said angle is less than 90°, then the longitudinal direction of the flow channel between the branch and the adsorption and/or absorption and/or filter device is preferably upwardly inclined, i.e., offset towards the inflow port.

The maximum flow cross section in the region between the branch and the adsorption and/or absorption and/or filter device can be less than or equal to the minimum flow cross section in the passage region. A tapering of this type is unproblematic in this case, because in any case only gases are supposed to flow through the adsorption and/or absorption and/or filter device.

For this purpose, according to which the region between the branch and the adsorption and/or absorption and/or filter device is supposed to serve only as a flow channel for gases, it can be provided according to the invention that the flow cross section in the region between the branch and the adsorption and/or absorption and/or filter device is constricted by a barrier with respect to the minimum flow cross section in the passage region, which barrier offers a greater resistance to at least solid substances, preferably also to liquid substances than gaseous substances.

Such a barrier is preferably configured as a tongue, which is arranged within the passage region at approximately the height of the branch.

The invention can be further developed that the barrier is configured as a tongue, which is fastened upstream of the branch and covers the branch at least in regions to the adsorption and/or absorption and/or filter device in the passage region in the case of the passage of stool or other excretions or secretions. A tongue of this type can be fixed either only on the upstream edge thereof or also on the two adjacent edges thereof.

The tongue can be configured to a limited extent to be bendable and/or elastic, and preferably consists of plastic. It should be connected along its fixed edges in such a way that it thereby maintains a preferred direction relative to the longitudinal direction of the passage region, which, thanks to a sufficiently rigid intrinsic quality thereof, is also always maintained independent of the orientation of the passage region, so that the branch is always kept open for gases. The actual purpose of the tongue consists of keeping passing excretions or secretions away from the adsorption and/or absorption and/or filter device, but at the same time permitting or encouraging the escape of gases to the adsorption and/or absorption and/or filter device. A small opening cross section in the region of a free edge of the tongue is sufficient for the removal of gas. This type of opening cross section could also be ensured by providing, on the rear side of the tongue, which faces the lumen branching off from the passage lumen, one or more nubs or other protuberances, which ensure a minimum distance of the tongue from the edge of the removal opening; naturally, these types of nubs could also be arranged in the region of the edge of the removal opening and when approaching the tongue enter into contact with it.

The tongue can be integrated with or connected to the hose-like or tube-like structure by adhesion or another joining technology.

It is preferred that the transition region between the tongue and the hose-like or tube-like structure is not configured to be articulated, but rather rigid so that the tongue always maintains its orientation in a predetermined direction.

The tongue can comprise one or more holes for the passage of gases.

Furthermore, the inlet opening of the adsorption and/or absorption and/or filter device, which inlet opening faces the passage region or the branch, can be covered by a gas-permeable, but preferably water-repellent separating layer. In this case, it is another measure to always keep the filter open for gases.

A preferred further development of the invention consists of the separating layer comprising a slightly wettable surface (lotus effect), in particular on the side thereof facing the passage region or the branch, so that the undesired adherence of excretions or secretions is prevented.

The separating layer can be configured for example as a microporous membrane, for example made of expanded polytetrafluorethylene, preferably with a maximum pore diameter of less than 1 mm, preferably with a maximum pore diameter of 100 μm or less, in particular with a maximum pore diameter of 10 μm or less.

On the other hand, the separating layer can also be produced from a paper, in particular from a waterleaf paper.

It lies within the scope of the invention that the adsorption and/or absorption and/or filter device comprises one or more surface-active substances that adsorb the odorous substances accumulating on the surface, and/or one or more absorbing substances that absorb the odor-active substances in the manner of a physical solution.

For example, the adsorption and/or absorption and/or filter device can include an adsorbing or absorbing or filtering granulate, such as activated carbon, for example in a quantity of 100 mL or less, preferably in a quantity of 50 mL or less, in particular in a quantity of 20 mL or less, or in a quantity of 10 mL or less, or even in a quantity of 5 mL or less.

It is recommended that the adsorption and/or absorption and/or filter device contains the granulate, for example the activated carbon, in a quantity of 2 mL or more, preferably in a quantity of 5 mL or more, in particular in a quantity of 10 mL or less, or in a quantity of 20 mL or more, or even in a quantity of 30 mL or more.

In order to guide the gas to be cleaned in a defined manner through the adsorption and/or absorption and/or filter device said device should comprise in the interior thereof one or more lamellar structures, which can assume the function of walls in a labyrinth.

An especially good cleaning or filtering effect is produced if the gas to be cleaned is guided on a route through the adsorption and/or absorption and/or filter device that is as long as possible. This can be achieved in particular in that a plurality of lamellar structures are offset against one another and/or are intertwined in the manner of interwoven fingers.

Another method of creating a route within the adsorption and/or absorption and/or filter device that is as long as possible for a gas that is to be cleaned consists of the adsorption and/or absorption and/or filter device comprising in the interior thereof at least one snail-shaped, spiral-shaped and/or helical structure so that the gas is forced to flow along said snail-shaped, spiral-shaped and/or helical structure.

The invention can be further developed to the effect that the adsorption and/or absorption and/or filter device comprises a material that filters infectious pathogens, in particular in the region of the downstream or outlet-side opening thereof. Due to a measure of this type, the transmission of diseases can be reduced.

If the adsorption and/or absorption and/or filter device is arranged in a replaceable cartridge or in a removable cap, after depletion of the adsorption and/or absorption and/or filter device, it can be replaced by a new one with a few hand movements.

The replaceable cartridge or the removable cap should comprise, in the region of the free front side thereof or the circumference thereof, one or more outlet openings, where the cleaned or filtered gas can escape into the environment.

The replacement of a used cartridge or cap is designed to be especially simple if the replaceable cartridge or the removable cap can be connected via a bayonet closure or via a screw closure to the hose-like or tube-like or T-shaped structure at the branch thereof.

Because the replaceable cartridge or the removable cap can be connected via the closure thereof optionally also to the inflow port of the hose-like or tube-like or T-shaped structure, it can be applied to the inflow port as an odor-safe closure for disposing of a filled collection bag.

In a further pursuit of the inventive idea, it can be provided that the hose-like or tube-like structure and/or the replaceable cartridge and/or the removable cap comprises at least one compartment for accommodating an aromatic substance. Because of these type of aromatic substances, it is possible, on the one hand, to improve the indoor climate; in this way, especially odors that have not been filtered out completely are thereby concealed, however.

The compartment for accommodating an aromatic substance can be open to the outside. However, so that the aromatic substance is released as much as possible only during use of the apparatus according to the invention, the compartment for accommodating an aromatic substance can be sealed with a removable cover, for example in the form of a peel-off foil.

The invention can be further developed to the effect that the compartment for accommodating an aromatic substance is configured to be annular and surround a central outlet opening of the adsorption and/or absorption and/or filter device in an annular manner.

As a part of an embodiment of this type, it is possible, inside the central opening of the annular compartment for accommodating an aromatic substance, for a wick or a non-woven-like and/or wide-meshed and/or large-pored fabric to be arranged, for example a wick stretched transversely over the central opening or a non-woven-like and/or wide-meshed and/or large-pored fabric, which extends into the compartment containing the aromatic substance for the moistening thereof with the aromatic substance.

Furthermore, the hose-like or tube-like structure can comprise for connecting a hose or collection vessel or collection bag or the like at the inflow port thereof and/or at the outflow port thereof a bayonet closure or a screw closure.

In addition, the hose-like or tube-like structure can be integrated with a collection vessel or collection bag, in particular in the region of the inflow port thereof.

On the other hand, it is also possible that the hose-like or tube-like structure comprises a hose or tube extension communicating from the outflow side thereof through the branch with the adsorption and/or absorption and/or filter device.

Such a hose or tube extension communicating with the adsorption and/or absorption and/or filter device can extend into a collection vessel or into a collection bag, in particular beyond a return-inhibiting foil there or another return-flow-inhibiting structure.

On the other hand, the hose-like or tube-like structure can be integrated with or can be connected to a head unit that is anchored or can be anchored in the rectum of a patient, which head unit can be anchored in the rectum via a balloon element that is placed or can be placed in the rectum.

The formation of odor can originate, in the case of the long-term use of a stool-removing system, moreover from the stool-removing hose arranged between the patient and the bag, wherein in particular the barrier property of the hose material that is used plays a decisive role. Conventional systems, which are normally made of silicone-based materials, are not suited for an odor-tight, continuous stool removal without an additional coating, such as example with parylene-based substances. The poor barrier properties of silicone are caused by the pronounced porosity of the silicones used. Even elaborate coatings of the silicone surface generally do not offer an adequate odor barrier effect. In the recent past, new types of silicone-based systems have been introduced that have a special, separately produced inner hose installed as a separate component that is made of a nylon-like or even Teflon-like material and thus make a substantially improved odor barrier possible.

To improve the odor seal, special cost-effective, PVC-based hose materials or hose materials with a coextruded PVC layer are being used as an alternative to silicone-based hose components. The barrier effect thereof is also adequately efficient for the majority of stools even without additional coating or inner hose technology.

The described apparatus for the decompression of intestinal gas and for the adsorption of odorous substances therefore is used preferably in conjunction with stool-removing hose lines that are PVC-based, contain PVC or are coextruded with PVC layers.

In order to minimize the diffusion of odorous substances through the supply or removal hoses, through a hose unit or head unit that is integrated or connectable therewith and/or through a collection bag that is integrated or connectable therewith, the hose-like or tube-like structure and/or a collection vessel that is integrated or connectable therewith and/or a collection bag that is integrated or connectable therewith and/or a hose unit or head unit that is integrated or connectable therewith can be made of a PVC-based material or of a material containing PVC or of a material that is coated or coextruded with one or more PVC layers.

The apparatus according to the invention can be structurally arranged in series or in parallel to the stool-removing system.

It is preferred that the hose-like or tube-like structure comprises a T-shaped form.

It is furthermore provided as a part of the invention that the removed stool or the removed excretions or secretions are conducted through the hose-like or tube-like or T-shaped structure of the apparatus in such a way that, in the case of a roughly normal vertical position of the apparatus, a contamination or an occlusion of the gas-discharging components of the apparatus is prevented to a great extent, wherein in particular accumulations of for example intestinal gas within the system can be prevented. The invention assumes that in the case of the drainage, the stool or the other excretions or secretions are conducted under the influence of gravity through the drainage line to the collection container. Therefore, a vertical alignment of the arranged is preferred for the operation of the system, and also a vertical alignment of the collection container. Precautions are taken for just this vertical alignment in order to prevent a contamination or an occlusion of the gas-discharging components of the apparatus to a great extent.

In order to ensure such a vertical alignment of the collection bag, the invention provides that a hook for vertically suspended fastening of the bag is provided on the hose-like or tube-like or T-shaped structure.

The invention can be further developed to the effect that the hook is configured to be widely projecting in such a way that, in the case of a collection bag that is laid flat, the hose-like or tube-like or T-shaped structure attached to the collection bag is thereby nevertheless upwardly inclined in the upstream direction, so that in spite of an alignment that is not completely vertical, the odor trap according to the invention functions.

Finally, it corresponds to the teaching of the invention that the hook is configured as a double-sided hook projecting in diametrically opposed directions or as a mounting bracket that can be rotated around the longitudinal axis of the hose-like or tube-like or T-shaped structure and can also be engaged as the case maybe in predetermined positions. Consequently, the side on which the collection bag is placed is irrelevant for functioning; the hose-like or tube-like or T-shaped structure attached to the collection bag always experiences an alignment with a sufficiently vertical component, in order to ensure the functioning of the odor trap. In the case of a double-sided hook that projects over both flat sides of the collection bag, the user is thereby relieved of all care concerning a sufficiently vertical arrangement; in the case of a rotationally adjustable hook, the user must make sure that the hook is swiveled towards the respectively lower flat side of the collection bag.

### BRIEF DESCRIPTION OF THE DRAWINGS

Further, structural and functional features of the odor-adsorbing, and/or filtering, and/or gas-removing unit according to the invention are yielded from the following description of preferred embodiments of the invention as well as based on the drawings which show:

FIG. 1a A lateral, sideward arrangement of the degassing unit for the stool-removing basic element of the apparatus.

FIG. 1b An embodiment of the apparatus according to the invention in the manner of a T-piece, wherein the decompressing and/or filtering unit is mounted in an reversibly detachable manner or even a fixed manner on the free end of the laterally outgoing leg or is connected to the outer wall of the leg.

FIG. 1c Another embodiment of the apparatus according to the invention in the manner of a T-piece, wherein the respective decompressing and/or filtering substance is integrated in a fixed design into the inner lumen of the lateral leg of the T-piece.

FIG. 2 An apparatus for the decompression, adsorption and/or filtering of intestinal gas that can be inserted into the stool-removing lumen of the system.

FIG. 3a An exemplary construction of a degassing unit arranged laterally to the stool-removing lumen, with gas guidance directed in a meandering manner and applied in a layer-like manner through the adsorber material and/or filter material.

FIG. 3b A special combination of a degassing element integrated in a fixed manner into the lumen of the basic element with an accessory unit, which can be replaced from bag to bag, with an enlarged adsorber or filter performance.

FIG. 3c A design of the apparatus according to the invention having spiral gas guidance through the adsorbing or filtering medium.

FIG. 4 An apparatus according to the invention which establishes a hose-like or tube-like gas-removing component or also, for example, one with a U-shaped profile, as a connection between the stool-receiving area of a collection bag and the gas-discharging unit that is upstream from the bag.

FIG. 5 Another embodiment of the apparatus according to the invention in which the gas-removing T-piece in a fixed design is installed in the inlet portion of a collecting vessel, and the adsorber function and/or filter function is integrated into a closure cap, which during the collection phase rests on the lateral leg of the T-piece and upon conclusion of collection is placed on the patient-side, upwardly pointing leg of the T-piece for a secure closure of the bag.

FIG. 6a An accessory adsorber and/or filter module, which releases fragrant or odor-neutralizing substances via a reservoir and dispenser component integrated into the housing.

FIG. 6b A dispenser module, which is placed on or is downstream in a direct extension from the gas-discharging opening of the apparatus, and releases odor-neutralizing aromatic substances via an integrated reservoir function.

FIG. 7 An embodiment of a double layer of foils acting valve-like in a flow directing manner; and

FIG. 8 An embodiment of a bag-like collection vessel.

### DETAILED DESCRIPTION OF THE PREFERRED EMBODIMENTS

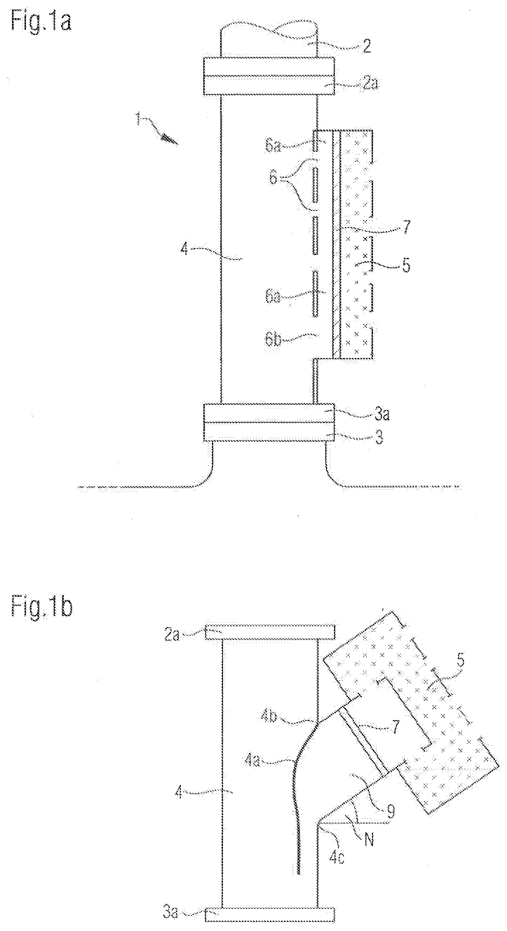

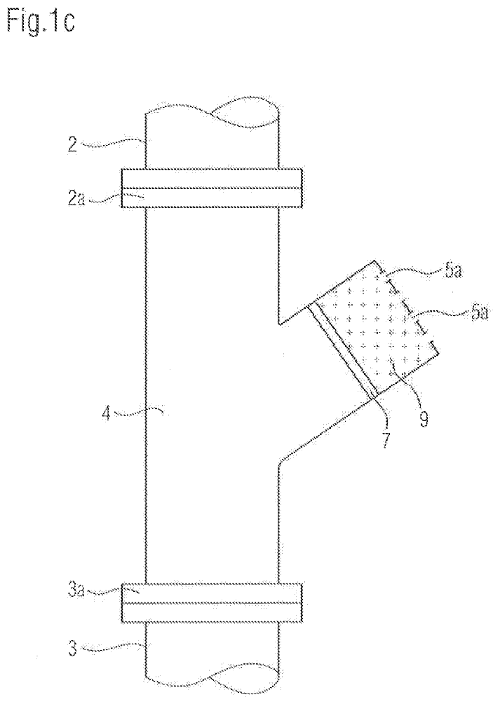

FIG. 1a shows a stool-removing apparatus 1 for removing intestinal gas to the environment, which apparatus is arranged as a separate, modular unit between the bag-side end 2 of the drainage line 12 and the stool-receiving inlet 3 of the collection bag 13. The apparatus 1 has, respectively terminally, connecting components 2a and 3a that are directed to the drainage line 12 and to the collection bag 13, with which components the apparatus 1 can be inserted into the stool-draining system in a serial arrangement, preferably in the region of the transition from the drainage to the collection bag 13 or to the stool-receiving container. A basic element 4 connects the connecting components 2a and 3a in such a way that said components are in contact with each other in a communicating manner.

Attached at the outer circumference of the wall of the tubular cylindrical basic element 4 of the apparatus is a degassing, adsorbing and/or filtering module 5 in a reversibly detachable form, or even in a form permanently connected to the basic element 4. The module 5 is nestled on the wall of the basic element 4 closed circularly as a collar, or even as a non-closed, C-shaped shell. The basic element 4 has, in the region of the connection of the filter module 5, one or more openings 6, which follow a gap-shaped intermediate space 6a, that points towards the adsorbing or filtering material and is bounded by a water-impermeable, but gas-permeable separating layer 7. The separating layer 7 can be made e.g., of a GoreTex-like material, which can be supplemented on the side facing the intermediate space 6a by a soil-repellent, for example lotus-like effect. Alternatively, a cost-effective, HME-filter-typical waterleaf paper can be used as a separating layer 7, which also acts in a water-repellent manner and at the same time is air-permeable. Conceptually, the side of the separating layer 7 facing the stool is preferably designed such that an exposure to stool does not lead to a long-term, gas-tight occlusion of the separating layer 7. Preferably on the lower end of the intermediate space 6a directed towards the bag 13, it transitions to an opening 6b in the stool-carrying or stool-removing area of the basic element 4. Stool that reaches the intermediate space 6a via the openings 6 can therefore flow via the opening 6b back into the lumen of the basic element 4. In a modification of this structural form, the stool-carrying lumen of the basic element 4 can also be connected to the intermediate space 6a solely via the opening 6b, in other words, not comprise any openings 6. The intermediate space 6a then extends from the opening 6b upwardly in a chimney-like manner, whereby the separating layer 7 lying above the opening 6b is protected from a direct stool exposure in the normal vertical position of the apparatus 1.

FIG. 1b shows an embodiment with a T-piece-like basic element 4 having a lateral leg 9 that is open toward the environment, to which the adsorbing and/or filtering module 5 is applied. In the preferred design of design of this structural form, the leg 9 is oriented upwardly in an angle of inclination N to the perpendicular or to the longitudinal axis of the basic element 4 or in the drainage direction towards upstream, of approx. 15 to 60 degrees, preferably 30 to 45 degrees. Therefore, in the case of a suspended, approximately normal vertical position of the apparatus 1 or of the unit of the stool-removing drainage hose 12, degassing apparatus 1 and collecting bag 13, the module 5 is protected from the direct influx of stool. Stool that has already penetrated into the leg 9 can drain from there, following the inclination of the leg 9, into the lumen of the basic element 4. As a supplement to the described inclination of the degassing leg, the transition to the stool-carrying lumen of the basic element 4 can be protected by a tongue 4a in the form of a curtain-like or canopy-like structure, in order to prevent the entry of stool into the degassing leg 9 even more efficiently. As shielding structure, the tongue 4a emerges, for example in the transitional region 4b between the laterally pointing leg 9 and the leg of the T-shaped basic element 4 running perpendicularly upwardly or towards upstream, from the inner wall of the basic element 4, and extends down beyond the lower transition 4c of the laterally pointing leg 9, into the leg of the T-shaped basic element 4 that runs perpendicularly downward or in the drainage direction towards downstream.

As an alternative to positioning a modular apparatus 5 between the drainage line 12 and collection bag 13, as a functional unit that can be connected serially if necessary, the basic element 4 can also be integrated in a fixed design directly into the upper inlet region 3 of the collection vessel 13. In the case of a bag-like container 13, the foil body thereof is connected with a tight seal by a direct welding or adhesion to the downwardly oriented leg of the T-piece that is pointing to the bag 13.

FIG. 1c shows an embodiment of the apparatus 1 based on FIG. 1b, in which the adsorbing and/or filtering material in a fixed design has already been integrated into the lateral gas-removing leg 9 of the T-piece-shaped basic element 4 that opens the entire system of the drainage 12 and bag 13 towards the environment. The leg is thereby dimensioned in such a way that it accommodates e.g., approx. 5 to 10 mL of activated carbon granulate. The carbon granulate is sectioned off from the stool-carrying, upper and lower legs of the T-piece-shaped basic element 4 by a gas-permeable, water-repellent separating layer 7. The front-side opening of the leg 9 is provided with a cap-like closure, which has openings 5a for the outlet of gas to the environment.

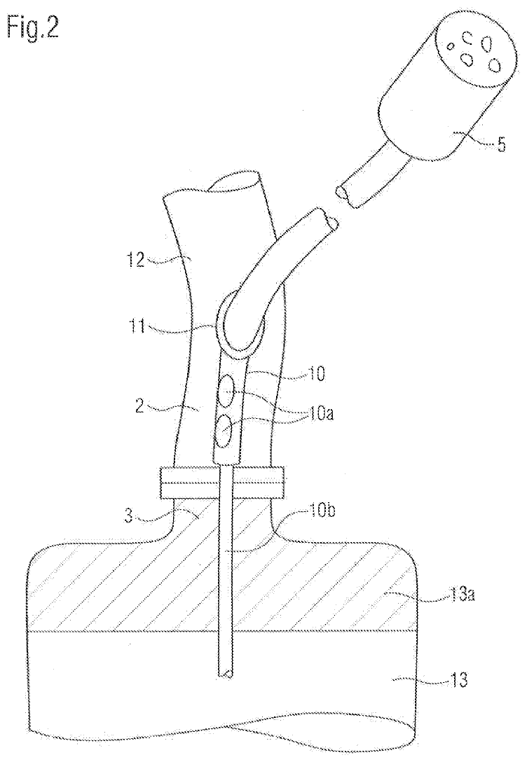

FIG. 2 shows a design of the apparatus 1, wherein the outlet of intestinal gas is established by a hose-like or tubular insert 10, which is inserted into the stool-removing unit. The insert 10 is inserted via an opening 11 designed with a custom fit, which in an unused state is sealed for example with a cap-like element. In the preferred embodiment, the distal end of the insert 10 is pushed forward into the stool-receiving internal area of the bag 13, wherein it potentially projects beyond return-inhibiting foil layers 13a, so that the foils do not impede the retrograde oriented removal of gas from the bag 13 and therefore can remove gas that has already reached the bag to the environment. The proximal end of the insert 10 terminally comprises a permanently attached adsorber and/or filter module 5. The insertion takes place optionally in the region 2 of the stool-removing catheter hose 12 that is close to the bag, preferably however in the region of the connection of the drainage catheter 12 to the collection bag 13, for example through an opening integrated into the respective connector or portions of the connector. In the preferred design, the insert 10 has large-lumen openings 10a as much as possible, which discharge above the return-inhibiting foils 13a in the stool-carrying area. The hose-like or tubular portion of the insert 10b, which extends beyond the return-inhibiting foils into collecting area of the bag 13, is preferably provided with a smaller lumen, in order to restrict a possible backflow of liquid content. The module 5 is positioned approx. 10 cm above the opening 11, in order to prevent a contamination from the bag contents in the case of the normal vertical position of the bag 13.

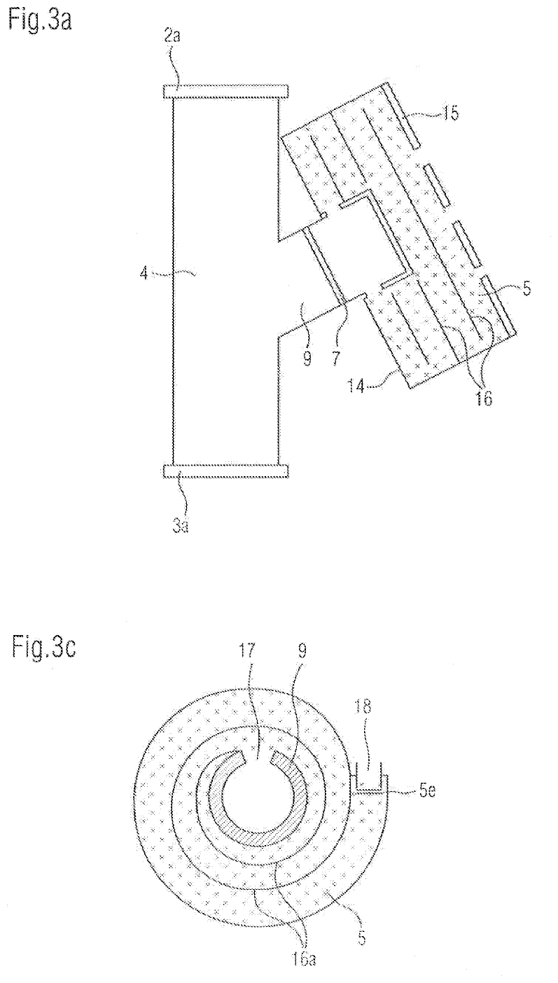

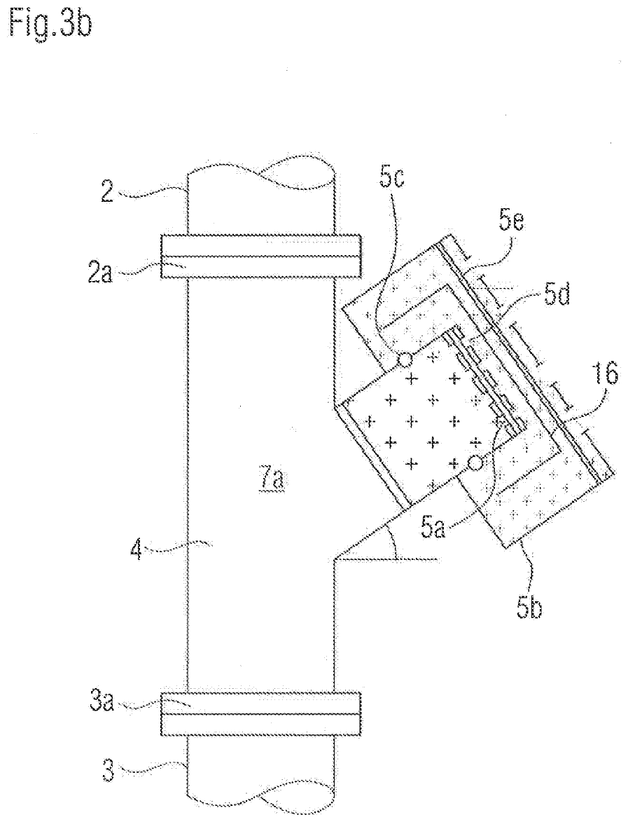

FIG. 3a shows an embodiment of the gas-discharging apparatus 1 corresponding to FIG. 1b with a T-piece-like basic element 4, wherein a module 5 with adsorber material and/or filter material is mounted on the lateral leg 9 with a tight seal. In the interior, the module 5 comprises lamellar structures 16, which lead the gas flowing out of the system gas as efficiently as possible, in the case of the best possible use of the odor-absorbing and/or filtering capacity, from the module base 14 to the module cover 15 through the module contents. The lamellae 16 can be for example horizontally offset, intertwined like fingers or even be guided in a spiral-shaped or helical manner. In the preferred case, the module 5 is arranged at the basic element 4 in such a way that the leverage that the module 5 exerts on the unit of the drainage catheter 12, degassing apparatus 1 and collecting bag 13 is kept as small as possible, and a forward tilting of the unit that is normally fastened in a suspended manner is thus prevented. The module 5 should have a correspondingly flat design that nestles the basic element 4 as much as possible and ideally is not offset from the center of gravity. The content of activated carbon in the module 5 of the described design should be at 20 to 100 mL, preferably at 30 to 50 mL.

FIG. 3b shows a combination of an adsorber or filter corresponding to FIG. 1a that is permanently built into the lateral leg 9, wherein an accessory, filtering and/or adsorbing filter cartridge 5b can be mounted in a reversibly detachable manner on the leg 9 as an additional unit. The cartridge 5b is provided to optimize the seal in the joining area between the leg 9 and the cartridge 5b with a sealing lip or an O-ring-like seal 5c. The gas escaping on the front side from the leg 9 is admitted via corresponding openings 5d in the bushing-like depression of the base of the cartridge 5b, and channeled through lamellar webs 16, in line with a most efficient possible adsorption through the adsorbing or filtering mass. In order to prevent the escape of microbes or viruses from the system into the environment, appropriate spore-proof, bacteria-tight and virus-proof filter material 5e is upstream of the outlet openings of the cartridge cover.

FIG. 3c shows a special form of the gas guidance through the filter material or the adsorber material, wherein the interior of the module 5 is provided with a spiral-shaped or snail-shaped separating wall 16a, which expands from the center or from the inlet opening towards the outside and opens terminally to the environment via another opening 17. To prevent the escape of infectious pathogens such as viruses or bacterial spores, an appropriate filtering material 5e is upstream of the opening 17.

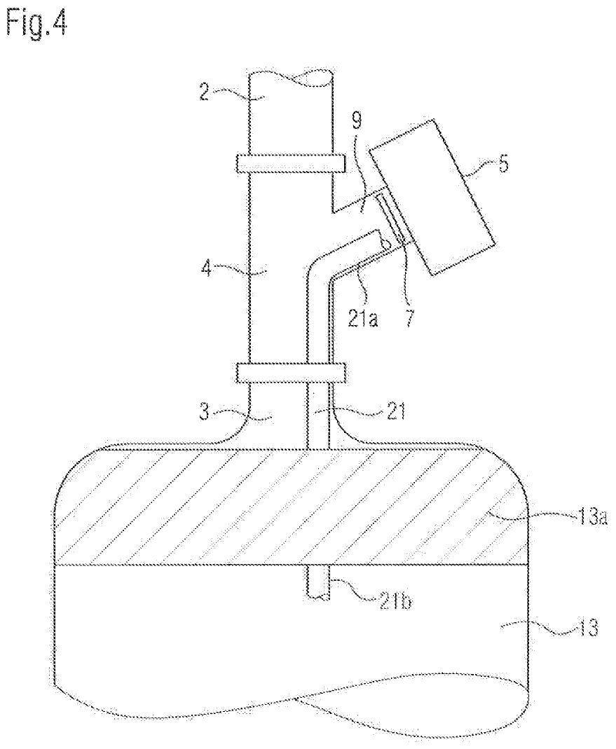

FIG. 4 shows a hose-like or tube-like, flexibly, or rigidly designed component 21, whose end 21a that is turned toward the patient is permanently attached at the inner wall of the basic element 4, preferably in the region of the lateral leg 9, and whose proximal end 21b is immersed into the stool-collecting area of the collection vessel 13 beyond the lower end of the basic element 4. The component 21 is thereby able overcome return-inhibiting components, such as for example lobe-like foil layers 13a, which fit closely with each other with a tight seal and act in a valve-like manner, so that intestinal gas that has penetrated into the bag 13 through the lumen of the component 21, and/or along the outer side of the component 21, can be emptied preferably directly into the gas-discharging leg 9 of the apparatus 1.

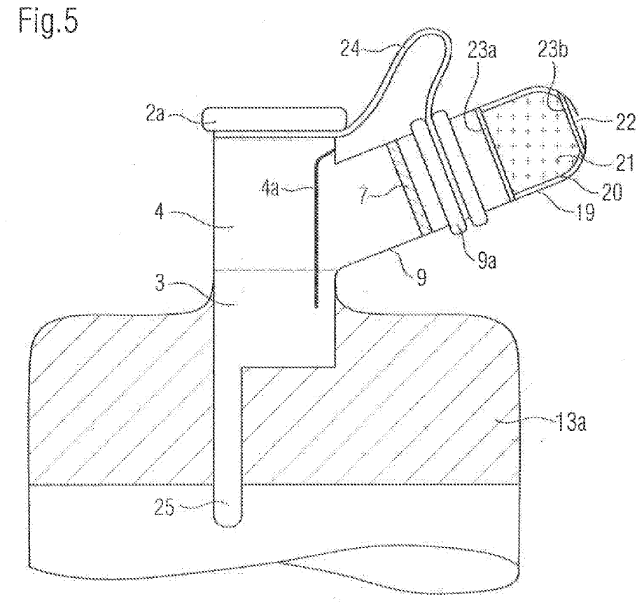

FIG. 5 shows a further structural form of an apparatus 1 according to the invention, wherein the T-piece-like basic element 4 is integrated permanently into the inlet portion 3 of the bag 13, preferably welded or adhesively secured. The lateral leg 9 is provided with a bayonet-like closure mechanism 9a, which corresponds to the closure mechanism 2a for connecting the drainage catheter 12 to the bag 13. During the phase of stool collection, the lateral leg 9 is closed with a closure cap 19. The cap 19 integrates a cartridge 20 with filter material 21 based on an activated carbon. Corresponding to the preceding designs, the free opening of the leg 9 is provided with a gas-permeable, but water-repellent and/or soil-repellent separating layer 7, which prevents the escape of stool from the T-piece-like basic element 4. The closure cap 19 is connected to the leg 9 in a gas-tight sealing manner. The cap 19 terminally comprises an opening 22 for the outlet of gas. As an alternative to a cartridge-like vessel, the internal area of the cap 19 can be filled directly with carbon or adsorber material or filter material, wherein the filled area on both sides 23a and 23b can be sealed for example with stable, but gas-permeable separating layers made of porous sintering material. In the case of using an activated carbon, the installed volume is 4 to 10 ml, preferably 5 to 6 mL.

If the bag 13 is disconnected from the drainage 12 and disposed of, the closure cap 19 is detached from the leg 9, is placed on the connector 2a and connected there with a tight seal. The separating layer 7 then prevents the free outflow of liquid through the lateral leg 9.

In order to impede the penetration of removed stool into the lateral leg 9, a tongue 4a protecting the transition from the stool-carrying path to the laterally branching leg 9 is also attached inside the T-piece-like basic element 4 in the form of a curtain-like or siphon-like structure.

In order to remove intestinal gas that has already penetrated into the collecting area of the bag 13, or in order to convey gas back into the T-piece-shaped basic element 4 upstream from the return inhibition via a return-inhibiting function integrated in the collecting bag 13, such as for example return-inhibiting foil layers 13a, and to conduct it from there through the adsorber and/or filter 5 to the environment, the T-piece-shaped basic element 4 has on the proximal end thereof a rod-like, tube-like or hose-like extension 25, which extends over a return-inhibiting foil or function into the collecting bag 13. The profile of the extension 25 is designed such that gas can be conducted either around the extension 25 or through the extension 25 from the collecting internal area of the bag 13 in regions of upstream of the bag 13. In the described structural form with a T-piece 4 integrated permanently into the inlet portion 3 of the bag 13, it is possible in an advantageous manner to dispense with an outlet that is commonly integrated directly into the bag wall in conventional collection bags 13. Outlet components that are integrated inside the collecting compartment into the wall thereof are especially problematic when the collection bag 13 gets into a flat horizontal position and the bag contents then potentially wet or saturate the filter whereby the filter loses its degassing function partially or completely.

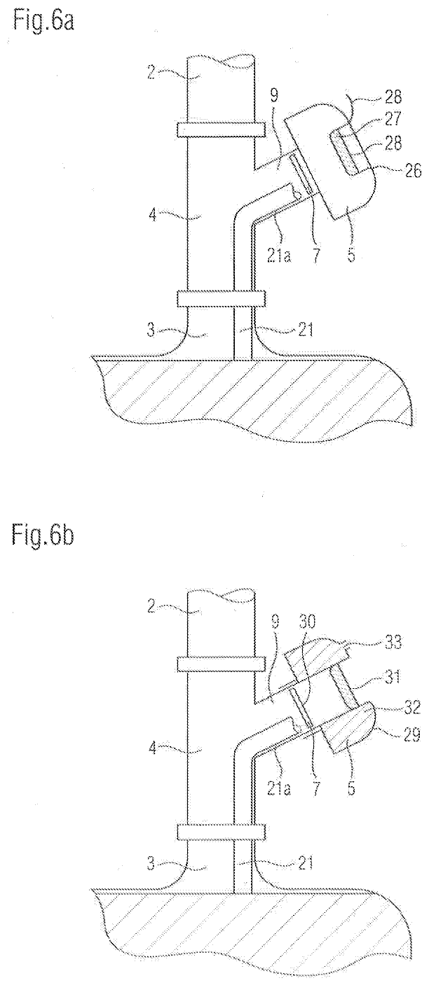

FIG. 6a shows a modified structural form of a module 5, 5b, which, besides an adsorbing and/or filtering effect, comprises a function that releases an aromatic substance. The release of substances that act in a perfuming manner or that mask unpleasant odors takes place from a compartment 26, which is integrated into the housing of the module 5, but is continuously separated from the adsorbing or filtering medium thereof. Inserted into the compartment 26 is an absorbent storage component 27 made for example of multi-layered pressed cellulose, which absorbs the aromatic substance over its correspondingly large total surface area and releases it in a delayed manner via its front surface pointing to the environment. The compartment 26 can be sealed with a peel-off foil 28. The release of an aromatic substance can also take place from a separate, for example capsule-alike container 28, which is inserted into a corresponding trough-like recess of the module 5. The respective aromatic substance can be selected by the user from a plurality of fragrance variants and be replaced.

FIG. 6b describes an accessory dispenser module 29, which is placed directly on the gas-discharging opening 30 of the apparatus 1 or of the T-piece-like basic element 4 or is arranged directly downstream from it, and continuously releases odor-neutralizing aromatic substances in the immediate outlet region of the apparatus 1 via an integrated substance-releasing function. In this special embodiment, intestinal gas that escapes from the apparatus 1 or the stool-draining entire system through the gas-discharging opening 30 passes through a non-woven-like, large-pored or wide-meshed fabric 31, which absorbs the aromatic substance in a similar manner as a wick from a reservoir component 32 and dispenses it via the correspondingly large surface of the porous or also meshed structure of the non-woven into the escaping gas and thus masks the odor-active ingredients thereof by means of correspondingly effective pleasant aromas.

The released aromatic substance is preferably based on volatile, oil-based solutions with sufficiently good running properties in the releasing non-woven. The reservoir 32 that is connected to the non-woven-like fabric 31 consists for example of a felt-like or sponge-like component, and can optionally be refilled with aromatic substance via a small-lumen opening 33 from the outside, for example through an ampule-like container that can be squeezed between the fingers, directly before the combination of the module 5 with the drainage apparatus 1 or with the T-piece-like basic element 4. With this system, the user can select between several fragrance variants. Furthermore, when switching the collection bag 13 or the entire system, the user can remove the module 5 from the bag 13 or from the system and transfer it to a new bag 13 or a new system. The module 5 can thus be used in the case of a patient over the total duration of use of the drainage apparatus 1. Once the reservoir is depleted, it can be filled again with aromatic substance through the opening.

Therefore, the invention describes an apparatus, which can be used as an accessory or be integrated permanently into a system for removing stool or other excretions or secretions, for absorbing and/or filtering gases, which collect for example in the course of a continuous closed stool removal within a stool-draining system, and wherein said gases are dissipated or decompressed without releasing unpleasant odors to the environment, wherein the apparatus is structurally arranged in series or in parallel to the stool-removing system, wherein the removal of the gas to the environment takes place outside of the collecting area of the collection vessel 13, and the respective gas-discharging component or function cannot be contaminated with the collected contents and consequently be rendered non-functional.

The removal of the gas to the environment preferably takes place directly above the collecting area, for example in the stool-removing hose of the system to the collecting area.

Gas that has already penetrated into the collection area of the vessel 13 can be removed, for example through a rod-like, tubular or hose-like extension in the region of the gas outlet and be decompressed from there.

The gas-discharging function of the filtering and/or absorbing material is maintained by a soil-repellent and/or water-repellent separating layer between the stool-removing internal area of the apparatus and the respectively connected filter module or absorber module 5.

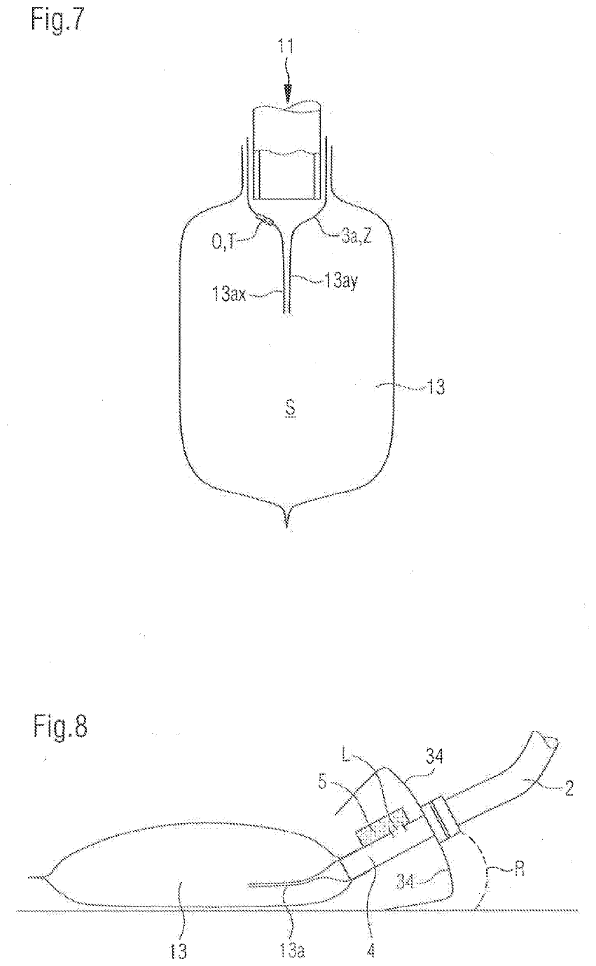

FIG. 7 shows a special design of a double layer of foils acting valve-like in a flow-directing manner, which double layer of foils allows a backflow of gas from the collection container 13 into the system supplying the bag.

FIG. 8 shows an angular holding apparatus for securing the bag in a vertically suspended position, wherein the backflow of the bag contents into the system supplying the bag is impeded in the case of a transient flat position of the bag.

FIG. 7 shows an arrangement of two foil layers 13ax and 13ay adjacent to each other in a planar manner and acting valve-like in a flow-directing manner, which are mounted in the interior of the collection container 13. Material M draining to the collection vessel enters through the two foil layers into collecting area S of the container. If the vessel fills up to the height of the two foil layers or beyond said foil layers when it is in the vertically suspended or normal upright position, the liquid contents cause a lip-like closure of the foil layers, and the undesired outflow or backflow of the contents from the collection container through the supplying opening is prevented. A corresponding effect occurs if gaseous substances get into the collection vessel. The gas filling likewise results in the tight seal of the two foil layers, whereby the gas accumulates successively in the vessel and the eventually resulting closing pressure on the two foil layers is pronounced in such a way that a further influx of liquid contents into the collecting vessel is no longer possible. Without a corresponding structural modification of the anti-blowback function, the collection vessel must be replaced in this case. In order for gas that has already penetrated into the bag to be removed from the bag, the invention provides specially designed foil layers, which even though they are impervious to liquid, they allow gas to pass through freely. The foil layers can be planar, e.g., consisting completely of an appropriate material, or also comprise the corresponding properties only proportionally in specific segments. The gas-permeable function or unit is placed advantageously there, where a complete closure of the two foil layers is precluded for mechanical reasons even in the case of maximum filling of the vessel. This is given in the region of the connection or the transition 3a of foil layers to a tubular or spout-like infeed segment or connector segment, where the sealing foil layers for example are welded on or adhesively secured. In the case of an outside diameter of the supplying element of approx. 15 to 25 mm, normally a gusset-shaped, open, non-closing region Z arises even in the case of the maximum filling of the vessel, which region comprises a height of at least 5 to 15 mm, and therefore offers sufficient area for the described function or integration of a “gas-permeable separating layer” T. As an alternative to gas-permeable foil material, a small, free opening O, of 1 to 2 mm diameter for example, can also be positioned in the gusset-shaped region Z. In the preferred case, however, a simple or cross-shaped slit can be made with a sharp blade in the described gusset-shaped region, wherein the legs of the slit comprise respectively a length of approximately 5 mm. Even though the slit cannot completely prevent the backflow of content into the segment above the collecting area, it does rule out a higher volume backflow of the already collected contents however.

FIG. 8 shows an embodiment of a bag-like collection vessel 13, wherein, in a horizontal position of the bag 13, the outflow of the bag contents into the supplying segment 4 of the bag 13 is impeded, reduced or prevented by means of a ramp-like slope R of the supplying segment 4. For this purpose, the angular hook 34 for the vertically suspended fastening of the bag 13 is designed in such a way that it brings the supplying or also the gas-discharging segment 4 into a diagonally ascending directed position relative to the flat horizontal bag 13. The resulting slope ensures that the liquid bag contents ideally do not reach the gas-discharging opening L in the supplying segment 4 of the collection bag 13 over a certain phase of the flat storage of the bag 13. The angular hook 34 is preferably designed to be double-sided and be fused on both flat sides of a bag 13 made for example of two layers, so that the desired inclination occurs in even in the case of the flat storage of the bag 13. In the preferred design, the hook 34 or the double hook is attached at a fixed 90 degree angle to the bag surfaces. The 90 degree position required for the inclination can also be achieved by an axially rotatable mounting bracket that can swivel into the 90 degree position and engage there.

List of Reference Numbers

| | |
|---|---|
| 1 | Apparatus |
| 2 | End |
| 2a | Connector |
| 3 | Inlet |
| 3a | Connector |
| 4 | Basic element |
| 4a | Tongue |
| 4b | Transition area |
| 4c | Transition |
| 5 | Module |
| 5a | Opening |
| 5b | Filter cartridge |
| 5c | Seal |
| 5d | Opening |
| 5e | Filter material |
| 6 | Opening |
| 6a | Intermediate space |
| 6b | Opening |
| 7 | Separating layer |
| 9 | Leg |
| 9a | Closure mechanism |
| 10 | Insert |
| 10a | Opening |
| 10b | Insert |
| 11 | Opening |
| 12 | Drainage line |
| 13 | Bag, collection vessel |
| 13a | Foil layer |
| 13a | Foil layer |
| 13a | Foil layer |
| 14 | Module base |
| 15 | Module cover |
| 16 | Lamella |
| 16a | Wall |
| 17 | Opening |
| 19 | Closure cap |
| 20 | Cartridge |
| 21 | Component |
| 21a | End |
| 21b | End |
| 22 | Opening |
| 23a | Side |
| 23b | Side |
| 25 | Extension |
| 26 | Compartment |
| 27 | Storage component |
| 28 | Peel-off foil, capsule |
| 29 | Dispenser module |
| 30 | Opening |
| 31 | Fabric |
| 32 | Reservoir |
| 33 | Opening |
| 34 | Hook |
| L | Opening |
| M | Material |
| N | Angle of inclination |
| O | Opening |
| R | Slope |
| S | Area |
| T | Separating layer |
| Z | Region |

1. An apparatus (1) for removing, in a manner that is as odor-free as possible, intestinal gas or other malodorous gases, as part of a system for the closed, preferably continuous, removal and/or collection of stools or other strongly odor-forming excretions or secretions from the human or animal body, wherein the stool or the removed excretions or secretions is or are guided outside the body through a hose-like or tube-like drainage line (12) downstream to a collection container (13), characterized by a hose-like or tube-like structure (4) inserted upstream of the collection compartment of the collection container (13), which structure, in addition to an inflow port and an outflow port and a passage region that connects said ports and is intended for the passage of stool or other excretions or secretions, has a branch (9) where intestinal gas or other malodorous gases are separated from the stool or the other excretions or secretions, and are conducted through an adsorption and/or filter device (5) to an outlet.

2. The apparatus (1) according to claim 1, characterized in that the branch (9) and/or the structure (4) comprising a branch (9) is arranged upstream of a return-inhibiting element at the inlet of the collection compartment of the collection container (13).

3. The apparatus (1) according to claim 2, characterized in that the return-inhibiting element comprises foil-based, lip-like or sail-like components (13ax,13ay) preferably wherein the distance between the foil-based, lip-like or sail-like components (13ax,13ay) tapers in the main flow direction, that is in the direction of the drainage line (12) to the collection compartment of the collection container (13).

4. The apparatus (1) according claim 1, characterized in that the adsorption and/or filter device (5) is spaced apart at least in sections from the passage region, in order to prevent, as far as possible, close contact with the stool or other excretions or secretions flowing through the passage region.

5. The apparatus (1) according to claim 1, characterized in that the longitudinal direction of the flow channel between the branch (9) and the adsorption and/or absorption and/or filter device encloses with the longitudinal direction of the passage region between the branch (9) and the outflow port an angle of 90° or less than 90°, for example an angle of 75° or less, preferably an angle of 60° or less, in particular an angle of 45° or less, or even an angle of 30° or less.

6. The apparatus (1) according to claim 1, characterized in that the maximum flow cross section in the region between the branch (9) and the adsorption and/or absorption and/or filter device (5) is less than or equal to the minimum flow cross section in the passage region.

7. The apparatus (1) according to claim 1, characterized in that the flow cross section in the region between the branch (9) and the adsorption and/or absorption and/or filter device (5) is constricted by a barrier with respect to the minimum flow cross section in the passage region.

8. The apparatus (1) according to claim 7, characterized in that the barrier is configured as a tongue (4a), which is arranged within the passage region at approximately the height of the branch (9).

9. The apparatus (1) according to claim 7, characterized in that the barrier is configured as a tongue (4a), which is articulated upstream of the branch (9) and covers the branch (9) to the adsorption and/or absorption and/or filter device (5) at least in regions with respect to the passage region in the case of the passage of stool or other excretions or secretions.

10. The apparatus (1) according to claim 8, characterized in that the tongue (4a) is configured to be bendable and/or elastic, and/or is preferably made of plastic.

11. The apparatus (1) according to claim 8, characterized in that the tongue (4a) is integrated with or connected to the hose-like or tube-like structure by adhesion or another joining technology.

12. The apparatus (1) according to claim 8, characterized in that the tongue (4a) is fixed via a rigid connection at the hose-like or tube-like or T-shaped structure (4).

13. The apparatus (1) according to claim 8, characterized in that the tongue (4a) comprises one or more holes (6) for the passage of gases.

14. The apparatus (1) according to claim 1, characterized in that the inlet opening of the adsorption and/or absorption and/or filter device (5), which inlet opening faces the passage region or the branch (9), is covered by a gas-permeable, but preferably water-repellent separating layer (7).

15. The apparatus (1) according to claim 14, characterized in that the separating layer (7) comprises a slightly wettable surface (lotus effect), in particular on the side thereof facing the passage region or the branch (9).

16. The apparatus (1) according to claim 14, characterized in that the separating layer (7) is configured as a microporous membrane, for example made of expanded polytetrafluorethylene, preferably with a maximum pore diameter of less than 1 mm, preferably with a maximum pore diameter of 100 μm or less, in particular with a maximum pore diameter of 10 μm or less.

17. The apparatus (1) according to claim 14, characterized in that the separating layer (7) from a paper, in particular from a waterleaf paper.

18. The apparatus (1) according to claim 1, characterized in that the adsorption and/or absorption and/or filter device (5) comprises one or more surface-active substances that adsorb the odorous substances accumulating on the surface, and/or one or more absorbing substances that absorb the odor-active substances in the manner of a physical solution.

19. The apparatus (1) according to claim 1, characterized in that the adsorption and/or absorption and/or filter device (5) includes an adsorbing or absorbing or filtering granulate, for example activated carbon, for example in a quantity of 100 mL or less, preferably in a quantity of 50 mL or less, in particular in a quantity of 20 mL or less, or in a quantity of 10 mL or less, or even in a quantity of 5 mL or less.

20. The apparatus (1) according to claim 19, characterized in that the adsorption and/or absorption and/or filter device (5) contains the granulate, for example the activated carbon, in a quantity of 2 mL or more, preferably in a quantity of 5 mL or more, in particular in a quantity of 10 mL or more, or in a quantity of 20 mL or more, or even in a quantity of 30 mL or more.

21. The apparatus (1) according to claim 19, characterized in that the adsorption and/or absorption and/or filter device (5) comprises in the interior thereof one or more lamellar structures (16) in order to guide the gas to be cleaned through the adsorption and/or absorption and/or filter device (5).

22. The apparatus (1) according to claim 21, characterized in that a plurality of lamellar structures (16) are offset against one another and/or are intertwined in the manner of interwoven fingers.

23. The apparatus (1) according to claim 19, characterized in that the adsorption and/or absorption and/or filter device (5) comprises in the interior thereof at least one snail-shaped, spiral-shaped and/or helical structure (16a) in order to guide the gas to be cleaned through the adsorption and/or absorption and/or filter device (5).

24. The apparatus (1) according to claim 19, characterized in that the adsorption and/or absorption and/or filter device (5) comprises a material that filters infectious pathogens, in particular in the region of the downstream or outlet-side opening (5a, 5d) thereof.

25. The apparatus (1) according to claim 1, characterized in that the adsorption and/or absorption and/or filter device (5) is arranged in a replaceable cartridge (5b, 20) or in a removable cap (19).

26. The apparatus (1) according to claim 25, characterized in that the replaceable cartridge (5b, 20) or the removable cap (19) is provided, in the region of the free front side thereof or the circumference thereof, with one or more outlet openings.

27. The apparatus (1) according to claim 25, characterized in that the replaceable cartridge (5b, 20) or the removable cap (19) can be connected via a bayonet closure or via a screw closure to the hose-like or tube-like or T-shaped structure (4) at the branch (9) thereof.

28. The apparatus (1) according to claim 27, characterized in that the replaceable cartridge (5b, 20) or the removable cap (19) can be connected via the closure thereof optionally also to the inflow port (2a) of the hose-like or tube-like or T-shaped structure (4).

29. The apparatus (1) according to claim 25, characterized in that the hose-like or tube-like or T-shaped structure (4) and/or the replaceable cartridge (5b, 20) and/or the removable cap (19) comprises at least one compartment (26) for accommodating an aromatic substance.

30. The apparatus (1) according to claim 29, characterized in that the compartment (26) for accommodating an aromatic substance is open to the outside and/or sealed with a removable cover, for example in the form of a peel-off foil (28).

31. The apparatus (1) according to claim 29, characterized in that the compartment (26) for accommodating an aromatic substance is configured to be annular and surround a central outlet opening (30) of the adsorption and/or absorption and/or filter device (5).

32. The apparatus (1) according to claim 31, characterized in that inside the central opening (30) of the annular compartment (26) for accommodating an aromatic substance, a wick or a non-woven-like and/or wide-meshed and/or large-pored fabric (31) is arranged, for example a wick stretched transversely over the central opening (30) or a non-woven-like and/or wide-meshed and/or large-pored fabric (31), which extends into the compartment (26) containing the aromatic substance for the moistening thereof with the aromatic substance.

33. The apparatus (1) according to claim 1, characterized in that the hose-like or tube-like or T-shaped structure (4) comprises at the inflow port (2a) thereof and/or at the outflow port (3a) thereof a bayonet closure or a screw closure for connecting a hose (12) or collection vessel or collection bag (13) or the like.

34. The apparatus (1) according to claim 1; characterized in that the hose-like or tube-like or T-shaped structure (4) is integrated with a collection vessel or collection bag (13), in particular in the region of the inflow port (3) thereof.

35. The apparatus (1) according to claim 1, characterized in that the hose-like or tube-like or T-shaped structure (4) comprises a hose or tube extension (21) communicating from the outflow side thereof through the branch (9) with the adsorption and/or absorption and/or filter device (5).

36. The apparatus (1) according to claim 35, characterized in that the hose or tube extension (21) communicating with the adsorption and/or absorption and/or filter device (5) extends into a collection vessel or into a collection bag (13), in particular beyond a return-inhibiting foil (13a,13ax,13ay) there or another return-flow-inhibiting structure.

37. The apparatus (1) according to claim 1, characterized in that the hose-like or tube-like or T-shaped structure (4) is integrated with or can be connected to a head unit that is anchored or can be anchored in the rectum of a patient, which head unit can be anchored in the rectum via a balloon element that is placed or can be placed in the rectum.

38. The apparatus (1) according to claim 1, characterized in that the hose-like or tube-like structure and/or a collection vessel that is integrated or connectable therewith and/or a collection bag (13) that is integrated or connectable therewith and/or a hose unit or head unit that is integrated or connectable therewith is made of a PVC-based material or of a material containing PVC or of a material that is coated or coextruded with one or more PVC layers.

39. The apparatus (1) according to claim 1, characterized in that the hose-like or tube-like or T-shaped structure (4) comprises a T-shaped form.

40. The apparatus (1) according to claim 1, characterized in that the apparatus is structurally arranged in series or in parallel to the stool-removing system.

41. The apparatus (1) according to claim 1, characterized in that the removed stool or the removed excretions or secretions are conducted through the hose-like or tube-like or T-shaped structure (4) of the apparatus in such a way that, in the case of a roughly normal vertical position of the apparatus (1), a contamination or an occlusion of the gas-discharging components of the apparatus is prevented to a great extent, wherein in particular accumulations of for example intestinal gas within the system can be prevented.

42. The apparatus (1) according to claim 1, characterized in that a hook (34) for vertically suspended fastening of the bag (13) is provided on the hose-like or tube-like or T-shaped structure (4).

43. The apparatus (1) according to claim 42, characterized in that the hook (34) is configured to be widely projecting in such a way that, in the case of a collection bag (13) that is laid flat, the hose-like or tube-like or T-shaped structure (4) attached to the collection bag (13) is thereby upwardly inclined in the upstream direction.

44. The apparatus (1) according to claim 42, characterized in that the hook (34) is configured as a double-sided hook projecting in diametrically opposed directions or as a mounting bracket that can be rotated around the longitudinal axis of the hose-like or tube-like or T-shaped structure (4) and can also be engaged as the case may be in predetermined positions.
